(19) United States
(12) Patent Application Publication
(10) Pub. No.: US 2008/0257343 A1
(43) Pub. Date: Oct. 23, 2008

(54) HINGELESS OXYGEN DELIVERY APPARATUS

(76) Inventor: LESLIE WILLIAM PETERSON, Castle Rock, CO (US)

Correspondence Address: SHERIDAN ROSS PC, 1560 BROADWAY, SUITE 1200, DENVER, CO, 80202, US

(21) Appl. No.: 12/106,509

(22) Filed: Apr. 21, 2008

Related U.S. Application Data
(60) Provisional application No. 60/912,867, filed on Apr. 19, 2007.

(51) Int. Cl. A61M 15/08; G02C 11/00; A61M 16/06

(52) U.S. Cl. 128/202.13; 128/207.18; 351/158

(57) ABSTRACT

The present invention relates to an apparatus used to deliver breathable gases to a person, which is integrally formed within the hollow frame of a pair of spectacles or eyeglasses, wherein oxygen is delivered from a supply or source directly to a user of the apparatus via the hollow frame of the spectacles or eyeglasses. The apparatus of the present invention is a hingeless, seamless, spectacle-shaped oxygen delivery system and delivery apparatus that is constructed, in whole or in part, of hollow tubular delivery members that are cast as a singe piece and rigidly connected to a base frame.

### CROSS-REFERENCE TO RELATED APPLICATIONS

The present application claims the benefit of priority, under 35 U.S.C. § 119, to U.S. Provisional Patent Application Ser. No. 60/912,867 filed Apr. 19, 2007 entitled “Hingeless Oxygen Delivery Apparatus,” the contents of which are incorporated herein in their entirety by this reference.

### BACKGROUND OF THE INVENTION

1. Field of the Invention

The present invention relates to an apparatus used to deliver breathable gases, such as oxygen, to a person who requires supplemental oxygen to assist in everyday life, wherein oxygen is supplied from an oxygen supply source to the nostrils of the person. More particularly, the present invention relates to an oxygen delivery apparatus which is integrally formed within the hollow frame of a hingeless pair of spectacles or eyeglasses, wherein oxygen is delivered from a supply source directly to the nose of a person via the hollow frame of the spectacles or eyeglasses.

2. Description of the Related Art

It is known to provide a hinged spectacle or eyeglass-based oxygen delivery apparatus, which is capable of continuously supplying oxygen to a person wearing the apparatus and which bears the appearance of a normal pair of eyeglasses. An example of a known apparatus is shown in FIGS. 1 and 2. As can be seen from these drawings, this apparatus has most of its frame constructed out of hollow tubular members in order to permit oxygen to flow through such hollow members from a source to the user. Specifically, the depicted apparatus has two temple members 2 and two corresponding end piece members 3 that are constructed of hollow tubular members, which are capable of communicating air from one to the other through a hollow, hinged connection 8. Each temple member 2 has an open end portion 6 that connects to a small-diameter supply cannula 4. The cannula 4 connects the open end portion 6 to an oxygen supply source (not shown). Similarly, each end piece member 3 extends around a lens of the eyeglass and terminates at an open end portion 7. Each end piece interconnects to a nasal cannula 5. In order for the oxygen to flow from the supply source through the delivery apparatus to the nasal cannulae 5, and thus to the nostrils of the user, the temple members 2 must be in fluid communication with the end piece members 3 through hollow hinged joints 8. During use, the temple members 2 of the known apparatus are unfolded so that a tapered or convex portion 12 of each end piece member 3 engages or mates with a flared or concave portion 14 of the corresponding temple member 2, thereby enabling oxygen to flow from the temple member 2 to the end piece member 3 and, ultimately, to the user. The force needed to disconnect the mated or interconnected members 12 and 14 is determined by an adjustable stop screw 10 (FIG. 2). The hinges 8 make it possible for the wearer to fold the apparatus when it is not in use, in a manner substantially identical to a typical pair of eyeglasses.

Notwithstanding the convenience folding brings to the user of the known delivery apparatus, the hinged design described above may be improved upon. For instance, in order for the known apparatus to be medically efficacious, it must be able to supply oxygen from the supply source, through the delivery apparatus, inclusive of the hinge, and to the nasal cannulae 5 at a reliable, regulated rate. Fluctuations in the rate of oxygen delivery can create anoxic conditions in the user, which may lead to medical problems. To ensure that the rate of oxygen remains as constant as possible from the supply source to the user, the apparatus must be air-tight, or as close to air-tight as is reasonably possible. As illustrated in FIG. 2, each of the end piece members 3 and the temple members 2 are constructed so that their respective diameters approximate, or are substantially equal to, the diameter of the supply cannulae 4. The supply cannulae 4 are typically small in diameter and this construction helps to minimize the weight of the apparatus. Similarly, the end piece 3 and temple members 2 are also small in diameter, which serves to lighten the apparatus and increase the comfort for the wearer. Moreover, the end piece members 3 and the temple members 2 are typically constructed with thin walls in order to achieve a lighter construction. As a result, the flared or concaved portion 14 of the temple member 2, into which the funnel-like portion 12 of the end piece member 3 is inserted has a thin-walled construction. During unfolding, the user of the known device inserts the funnel-like portion 12 of the end piece member 3 into the concave portion 14 of the temple member 2. Each time this happens, frictional contact occurs between these two structures. After repeated folding and unfolding, this frictional contact has a tendency to rub the thin contacting surfaces of the funnel-like portion 12 and the concave portion 14, creating areas of wear which create locations where oxygen may escape. Over time, these areas of wear can grow such that gaps are formed between the funnel-like portion 12 and the concave portion, creating locations where oxygen can escape during use. Gaps of significant size or number can result in a medically significant loss of oxygen to the wearer of the delivery apparatus.

In addition to frictional contact, after repeated folding and unfolding, the stop screw 10 of the hinged joint 8, through which the end piece member 3 is rotatably connected with the temple member 2, may become loosened. When this happens, the alignment and point of contact between the open end portion 14 and the funnel-like portion 12 can be compromised, offsetting the hinged connection. When an apparatus in this condition is unfolded, the funnel-like portion 12 may not properly next with the open end portion 14, creating a danger that funnel-like portion 12 will become deformed or bent during opening, as the user may apply more force than is necessary to open the apparatus or because the funnel-like portion 12 will contact the open end portion 14 in a mis-aligned manner. There is thus a danger that the funnel-like portion 12 may become bent and therefore impede the flow of oxygen to the wearer or create a leak diverting the supply of oxygen away from the wearer.

Additionally, in many known delivery devices, the hollow portions of the eyeglass frame which serve to carry oxygen from the supply source to the user are cast in two halves and subsequently joined to form a hollow structure in the finished product. Therefore, these known devices include seams along at least a portion of the oxygen delivery route. As mentioned above, the walls of many known apparatuses are thin in order to reduce weight, provide as much comfort to the wearer as possible, and reduce material costs. Because of this, and because no method of connecting the two cast halves together is infallible, over time the seal between the two halves can loosen through repeated use and normal wear and tear, or may be unintentionally bent at the seam, creating leaks and areas of structural weakness. As before, this can cause a loss of oxygen from the point of delivery to the user, which may lead to medically anoxic conditions.

While the foregoing describes what is generally known, nothing herein is to be construed as an admission that the present invention is not entitled to antedate a patent, publication or invention by another by virtue of prior invention.

### SUMMARY OF THE INVENTION

The present invention provides an improved oxygen delivery apparatus that overcomes the aforementioned problems that are inherent in the prior art. Embodiments of the present invention provide a hingeless, eyeglass-shaped oxygen delivery system and apparatus that is constructed, in whole or in part, of hollow tubular delivery members that are cast as a singe seamless piece and then rigidly connected to a base frame in order to create an apparatus that may be worn by a user in a manner identical to a typical pair of eyeglasses. By removing the hinges and casting the hollow members as a single seamless piece, the present invention reduces and prevents the creation of leaks through repeated use and normal wear and tear. It is also an object of the present invention for the hollow tubular delivery members to be thin-walled in construction so as to be as lightweight and comfortable for the user, and made of a material that is sufficiently bendable or formable to allow for customization in fit, yet structurally sound such that a moderate amount of bending or forming will not compromise the flow of oxygen to the user.

The above objects are accomplished by the present invention which, in several embodiments, provides an oxygen delivery apparatus having an eyeglass or spectacle shaped delivery system, wherein the apparatus is constructed of a plurality of eyeglass frame members, some of which are hollow tubular members capable of allowing a fluid flow of oxygen therethrough, and some of which are solid, structural members that provide stability to the apparatus. In some embodiments, the frame members include a single base frame in the shape of a pair of templeless eyeglasses, which is capable of housing lenses positioned in front of the wearer's eyes, and two hollow, tubular temple members, each of which includes nose rests or nose pads. The base frame is configured such that the two temple members can be attached to the top and/or back side of the base frame. When the two temple members are attached to the base frame in this manner, the apparatus is structurally similar to a typical pair of eyeglasses. Therefore, when fully assembled, the oxygen delivery apparatus of the present invention is wearable by the user in a manner identical to a pair of eyeglasses.

In some embodiments, each temple member is cast individually as a single, continuous, hollow tube with no seams or hinges. They are also cast such that, when the apparatus of the present invention is worn by a user, the tubular temple members extend from the back of the wearer's ears forward to the temple area, in a manner similar to a typical reach on a pair of eyeglasses, then turn inward along the upper portion of the base frame toward the center of the face of the wearer, where they turn downward, along the inner edges of the two lenses and along either side of the bridge of the user's nose, and terminate adjacent to a pair of nose pads. One tubular temple member is cast for the left side of the apparatus and the other is cast for the right side, such that the left and right temple members are mirror images of each other. Both of the tubular temple members have a distal open end portion configured to be connected to a cannula, which extends from the distal open end portion of each temple member to an oxygen supply, and a proximal open end portion configured to be connected with a nasal cannula, which extends from the proximal open end portion of each temple member to the corresponding nostril of the user. One end of each supply cannula is connected to a temple member at its distal end, behind the user's ears; the other end is connected to an oxygen supply, such as a direct line feed or a tank. The nasal cannulae each connect to a proximal end of the temple members at the user's face at a location generally near the nose pads and extend from the proximal ends directly into the user's nostrils. Each temple member is attached to a base frame, which is cast separately from the temple members, such that the final configuration of the inventive apparatus resembles a typical pair of eyeglasses. The tubular temple members are configured such that they allow for the fluid flow of oxygen through them with no interruptions or breaks in the path of oxygen flow and thus with no reduction in supply or pressure. With the supply and nasal cannulae in place as described, the flow of oxygen proceeds from the oxygen source to and through the supply cannulae and into the distal ends of the temple members. From there, the oxygen passes through the hollow, tubular temple members, into and through the nasal cannulae, and directly into the user's nostrils.

In other embodiments, the oxygen delivery apparatus is constructed so that the base frame and the hollow, tubular temple members are cast as a single article, not separate pieces. In this embodiment, at least a portion of the base frame is comprised of at least a portion of each hollow temple member, such that the base frame is no longer a separate piece. As with the previous embodiment, each of the hollow temple members has a distal open end portion configured to be connected to a supply cannula, and a proximal open end portion configured to be connected to a nasal cannula. The supply cannulae are also connected to an oxygen supply, such as a direct line feed or a tank, and the nasal cannulae are connected to the distal end of the temple pieces and serve to deliver oxygen directly to the user. The tubular temple members are configured such that they allow for the fluid flow of oxygen through them with no interruptions or breaks in the path of oxygen flow and thus with no reduction in supply or pressure.

Although well suited for use in the delivery of oxygen to users, and although much of the discussion of the present invention is directed toward the delivery of oxygen, one of ordinary skill in the art will appreciate that advantages offered by embodiments of the present invention may be realized in the delivery of numerous breathable gases to users, including, without limitation, oxygen, air, compressed air, nitrous oxide, nitrogen, and other breathable gases, as well as any combination thereof.

These and other advantages will be apparent from the disclosure of the invention(s) contained herein. The above-described embodiments and configurations are neither complete nor exhaustive. As will be appreciated, other embodiments of the invention are possible using, alone or in combination, one or more of the features set forth above or described in detail below.

Various embodiments of the present invention are set forth in the attached figures and in the detailed description of the invention as provided herein and as embodied by the claims. It should be understood, however, that this Summary does not contain all of the aspects and embodiments of the present invention, is not meant to be limiting or restrictive in any manner, and that the invention as disclosed herein is and will be understood by those of ordinary skill in the art to encompass obvious improvements and modifications thereto.

Additional advantages of the present invention will become readily apparent from the following discussion, particularly when taken together with the accompanying drawings.

### BRIEF DESCRIPTION OF THE DRAWINGS

FIG. 1 is a perspective view of a known oxygen delivery apparatus;

FIG. 2 is an enlarged perspective view of the hinged joint of the known oxygen delivery apparatus shown in FIG. 1;

FIG. 3 is a perspective view of an oxygen delivery apparatus according to at least some embodiments of the present invention;

FIG. 4 is an exploded view of an oxygen delivery apparatus according to at least some embodiments of the present invention;

FIG. 5 is a side perspective view of an oxygen delivery apparatus according to at least some embodiments of the present invention; and

FIG. 6 is a close-up perspective view of the oxygen delivery apparatus depicted in FIG. 5.

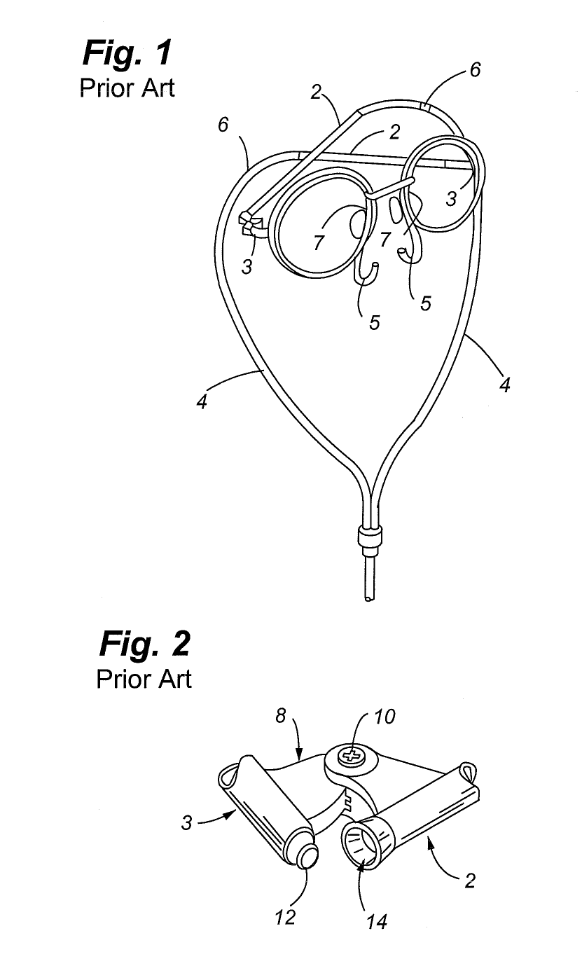

The drawings are not necessarily to scale and the drawings may include exaggerated features for purposes of clarity.

### DETAILED DESCRIPTION OF THE PREFERRED EMBODIMENTS

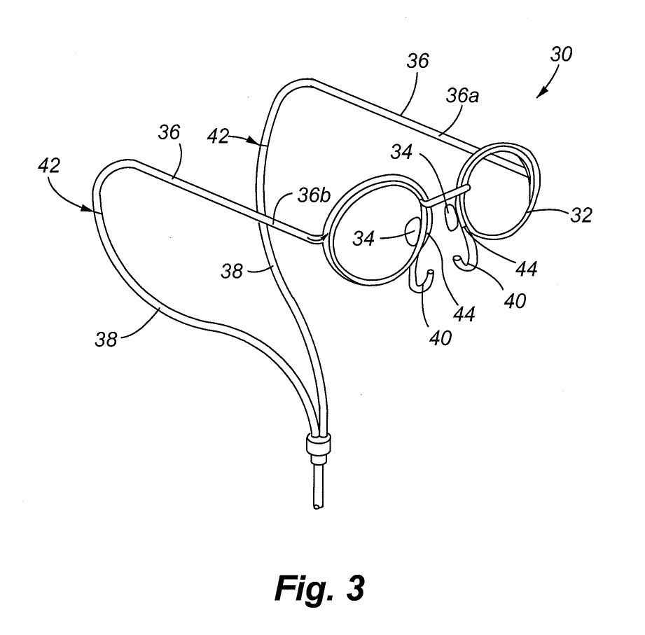

Referring initially to FIG. 3, an oxygen delivery apparatus 30 in accordance with at least one embodiment of the present invention is presented. The apparatus 30 is constructed of a plurality of eyeglass or spectacle frame components, including a base frame 32 and two hollow tubular temple members 36. Each hollow tubular temple member 36 has a nose pad 34 attached to it and each is configured to fit on opposite sides of the base frame 32 upon assembly of the apparatus, such that each are mirror-image representations of each other (e.g. a first hollow tubular temple member is configured to fit on the left side of the apparatus and a second hollow tubular temple member is configured to fit on the right side). The temple pieces 36 are otherwise structurally and functionally identical. The tubular temple members 36 are configured to attach to opposing sides of the base frame 32 and the base frame 32 is configured to receive the temple members 36, accordingly. As can be seen in the depicted embodiment, when the temple members 36 are attached to the base frame 32, one temple member 36a is attached to the left side of the base frame 32 and the other temple member 36b is attached to the right side of the base frame 32. The fully assembled apparatus 30 thus resembles a typical pair of eyeglasses in structure and function and is worn by the user as eyeglasses when in use, with at least a portion of the left temple member 36a contacting at least a portion of the left ear of the user and at least a portion of the right temple member 36b contacting at least a portion of the right ear of the user. Similarly, when in use the nose pad 34 of the left temple member 36a contacts the left side of the nose of the wearer and the nose pad 34 of the right temple member 36b contacts the right side of the nose of the wearer, in a manner similar to the nose pads of a typical pair of eyeglasses. In some embodiments, the apparatus 30 optionally includes a pair of supply cannulae 38, a pair of nasal cannulae 40, and an oxygen supply or source (not shown).

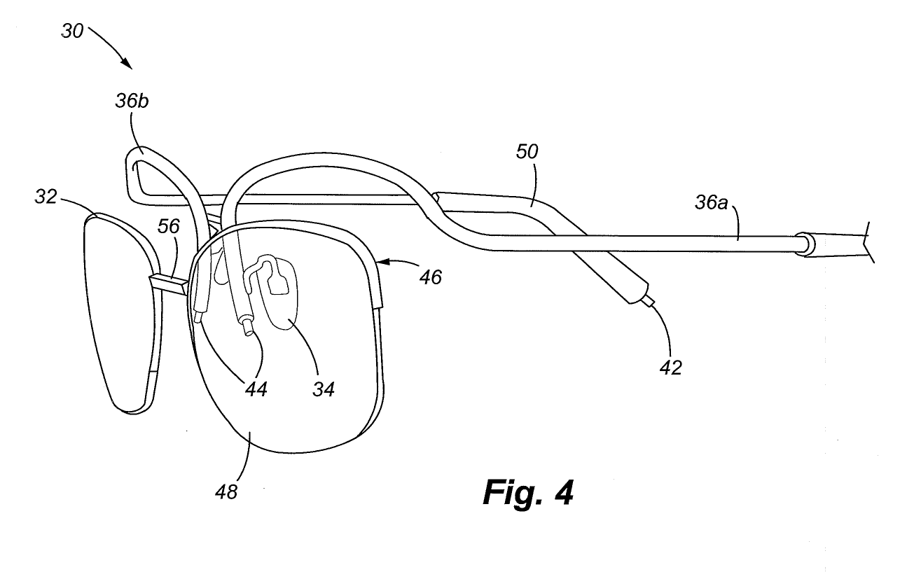

Referring now to FIG. 4, an exploded view of an oxygen delivery apparatus 30 in accordance with at least some embodiments of the present invention is provided. The left temple member 36a and the right temple member 36b are both configured to attach to opposing sides of the base frame 32, with the left temple member 36a attached to the left portion of the back side 46 of the base frame 32 and the right temple member 36b attached to the right portion of the back side 46 of the base frame 32. Additionally, when fully assembled, the temple members 36 are configured to attach to the base frame 32 such that they only contact the back side 46 of the base frame 32 and do not cover any portion of the lenses 48, if present, or block the wearer's field of vision. The temple members 36 are also configured so that, when the apparatus 30 is in use and being worn by a user, the temple members 36 contact the user's nose and ears, similar to a typical pair of eyeglasses. Specifically, each of the temple members 36 contact the user's nose via the nose pads 34 at approximately the bridge of the user's nose and each contacts the user's ears via the ear rests 50, at approximately the point of attachment of the auricle of the user's ear to the user's head. When the apparatus 30 is assembled, the combination of the base frame 32 and temple members 36 provide structural stability to the apparatus 30. This stability prevents the temple members 36 from shifting during use and also serves to hold the temple members 36 in place so that the oxygen delivery apparatus 30 of the present invention is wearable by a user in a manner identical to a pair of eyeglasses.

Both of the temple members 36 are hollow in construction and are preferably formed as a single, seamless piece of material. Additionally, both of the temple members 36 have a distal open end portion 42, which is located at a point behind the user's ear when the apparatus 30 is being worn by a user. Each distal open end portion 42 is configured to be in fluid communication with a supply cannula 38, such that the supply cannula 38 provides the means by which oxygen may be delivered from a supply source to the proximal open end portion 42 and into the interior chamber of the hollow temple member 36. Additionally, each of the temple members 36 has, at the end opposite from the distal open end portion 42, a proximal open end portion 44, which is located at a point alongside the user's nose when the apparatus 30 is being worn by a user. Each proximal open end portion 44 is configured to be operatively connected with a nasal cannula 40, such that the nasal cannula 40 provides the means by which oxygen may be delivered from the hollow interior chamber of the temple member 36, to the proximal open end portion 44, and into the nasal cannula 40. The points of connection between the distal open end portion 42 of each temple member 36 and the supply cannulae 38, and between the proximal open end portion 44 of each temple member 36 and the nasal cannulae 40, are configured to be air-tight or nearly air-tight, and preferably completely hermetic, so that the flow of oxygen from the supply, through the apparatus 30, and to the user is reliable and as constant as possible.

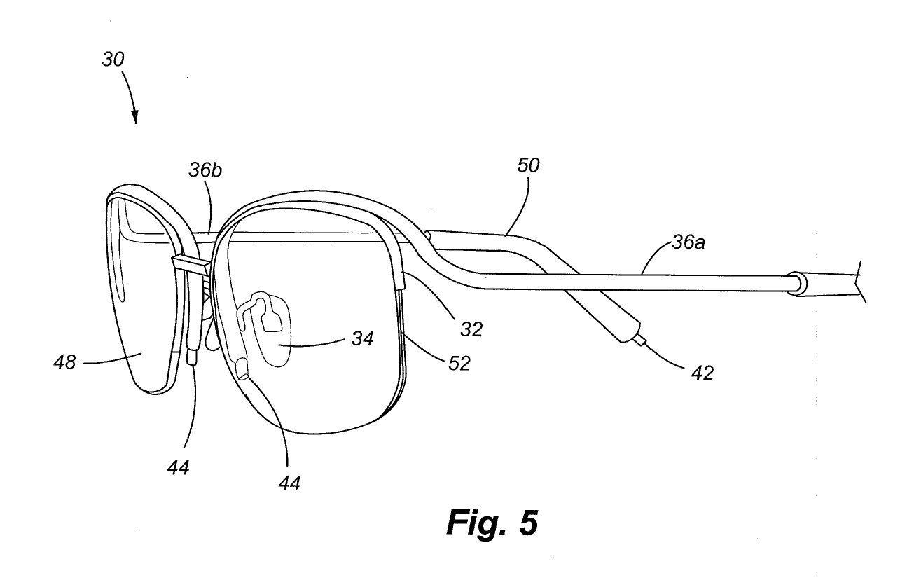

In some embodiments, the base frame 32 is a separate frame component that is configured to support a pair of lenses 48. A bridge piece 56 interconnects the lens supporting portions of the base frame 32. While the base frame 32 is configured to house lenses 48, lenses are not required for operation of the apparatus 30 of the present invention. Some users may not need or desire lenses but nonetheless prefer this type of oxygen delivery system. As shown in FIGS. 3 and 4, in those embodiments where the base frame 32 is constructed to include lenses 48, the base frame 32 may be configured to completely encircle the lenses 48 (FIG. 3), or to partially encircle the lenses 48 (FIG. 4), to accommodate a variety of eyeglass styles and to house a variety of shapes and sizes of lenses 48. The lenses 48 may be secured in place in the base frame 32 via any number of standard means 52 (FIG. 5) typically used in eyewear including, without limitation, being completely housed within a portion of the base frame 32 or being at least partially housed within a portion of the base frame 32, with the balance of the lens held in place by wire, plastic threading, screws, rivets, bolts, pins, or similar means. The base frame 32 may be configured to house a variety of shapes of lenses 48 and is thus sufficiently scalable so as to be able to house lenses 48 of virtually any shape or size and thus be able to conform to eyeglass fashion as it changes from time to time without impairing the structural stability of the base frame 32 or the apparatus 30 itself and without impeding or hindering the fluid flow of oxygen through the apparatus 30 in any manner.

The lenses 48 may be corrective or non-corrective, and may even be omitted in some configurations, though the presence of the lenses 48 in some embodiments serves to increase the structural stability of the base frame 32 and thus the oxygen delivery apparatus 30 as a whole. Preferably, the base frame 32 does not serve as part of the oxygen delivery system, but rather serves as a means for positioning the temple members 36 and the lenses 48, as well as providing the structural foundation for the eyeglass-like appearance of the present invention. It is therefore preferable for the base frame 32 to not be constructed of hollow members, but rather to be constructed as a solid structure. Preferably, the base frame 32 is made of a material that grants it sufficient structural stability as well as resistance to the damage and/or wear that can result from regular use, and that is sufficiently light weight so as to afford the maximum amount of comfort to the user of the apparatus 30. It is also preferable that portions of the base frame 32 are bendable or formable so as to allow the user to customize the fit of the apparatus 30 to his or her face, without compromising the structural integrity of the base frame 32, the fully assembled apparatus 30, or the path of oxygen delivery.

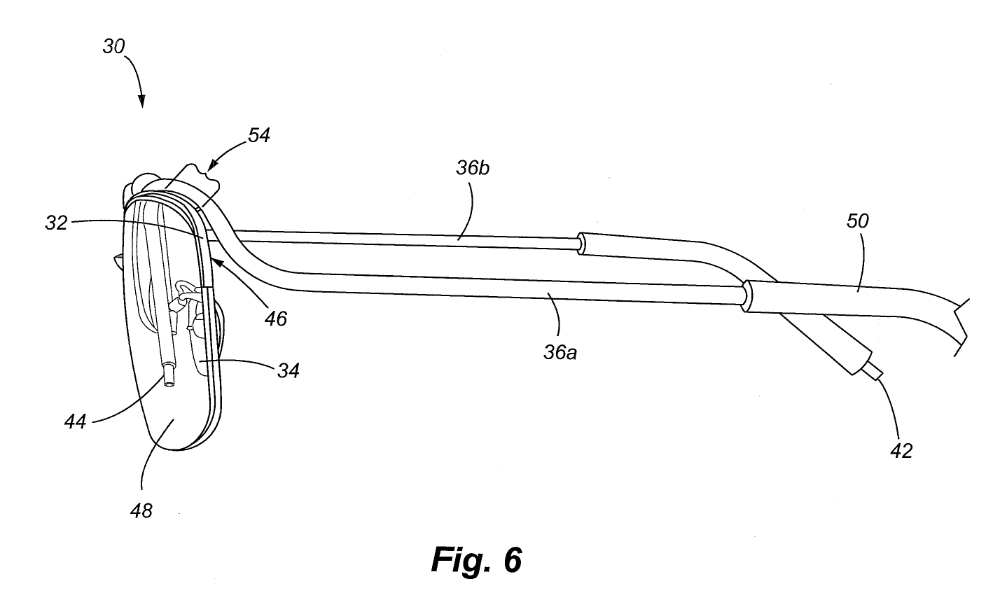

As shown in FIG. 6, in some embodiments the base frame 32 is configured to be the point of attachment for the temple members 36. The temple members 36 are attached to the base frame 32 via at least one, and preferably a plurality, of attachments 54 located along the back side 40 of the base frame 32. The total numbers of attachments 54 may vary between embodiments to suit the strength of the connection desired. FIG. 6 illustrates a single attachment 54 on one side of the base frame 32. Preferably, there will be attachments 54 that are identical in number and location on both sides of the base frame 32 so that the apparatus 30 will have a generally balanced or symmetrical appearance. Thus, in the depicted embodiment, there are two attachments 54, one on each side of the base frame 32. Those of skill in the art will appreciate that the temple pieces 36 may be attached to the base frame 32 at locations different from those depicted and with a greater or lesser number of attachments 54. The attachments 54 are sufficiently strong so as to permanently join the base frame 32 to the temple members 36 and to resist moving or rotating of the temple members 36 when the apparatus 30 is in use. Therefore, the total number of attachments 54 may vary, depending upon the strength of connection needed to accomplish this goal. Preferably, the total number of attachments 54 ranges from 1 to 8, more preferably from 2 to 6, and even more preferably the total number of attachments 54 is four, with two located on each side of the base frame 32. The attachments 54 may be created by any number of standard means of permanently joining two physical objects together. By way of example and not limitation, such means may include welding, spot welding, soldering, adhesives, screws, pegs, rivets, and similar means of attachment. Preferably, the attachments 54 are created by soldering. The attachments 54 may be small, such as a dot, point or spot connection, or the attachments 54 may be large and have a length that is measurably different from a single point of connection.

The hollow tubular temple members 36 provide the means by which the apparatus 30 delivers oxygen from a supply or source to a user. Preferably, the temple members 36 are cast as a single piece and therefore lack any seams or hinges of any kind. By casting the temple members 36 as a single piece, rather than as two or more separate pieces that are subsequently pieced together, the temple members 36, and thus the delivery apparatus 30 of the present invention, are resistant to the creation of leaks and points of wear that can occur with the presence of hinges and seams, such as those that are present in several of the known devices. The path of oxygen delivery of the present invention is therefore uninterrupted from the supply source to the wearer, because the temple pieces 36 do not contain any hinges, points of rigid connection such as seams, or other areas along the path of oxygen delivery where leaks may develop. As with the base frame 32, the temple pieces 36 are preferably made of a material that provides sufficient structural stability and rigidity so as to be able to function as eyeglasses, to effectively deliver oxygen from the supply to the user, and to resist the deterioration or wear that comes from regular use. It is also preferable for the temple pieces 36 to be sufficiently light in weight so as to afford the maximum amount of comfort to the user of the apparatus 30. Preferably, the temple pieces 36 are made of a material that provides each of the foregoing features, but that is also sufficiently bendable or formable to allow the user to customize the fit of the apparatus 30 to his or her face, without impeding the flow of oxygen through the hollow interior of the temple members 36 or compromising the integrity of the oxygen delivery apparatus 30.

Preferably, the supply cannulae 38 are connected to distal open end portions 42 of the temple pieces 36 at locations behind the user's ears when the apparatus 30 is in use. To facilitate this connection, the distal open end portions 42 of the temple pieces 36 are configured to provide a point of attachment for the supply cannulae 38, with the cannulae 38 fitting snugly over points of connection at the distal open end portions 42 such that a fluid connection is created between the cannulae 38 and the distal open end portions 42. In some embodiments, the points of connection at the distal open end portions 42 are modified portions of the distal ends of the temple pieces 36 having a decreased wall thickness. In other embodiments, the points of connection at the distal open end portions 42 are short, tubular inserts that are of a diameter such that they may be partially inserted into the distal open end portions 42, leaving a portion of them outside of the distal open end portions 42. These short, tubular inserts may be permanently attached to the distal open end portions 42 by any number of standard means, including without limitation, welding, soldering, adhesives, or other means described herein. Alternatively, the short, tubular inserts may be removable from the apparatus 30 such that they may be replaced when they become worn. In either embodiment, the modified portions of the distal ends of the temple pieces 36 or the short, tubular inserts provide points of decreased diameter at the terminal points of the oxygen supply conduit of the temple pieces 36, over which first ends of the supply cannulae 38 may be fitted to create a path of oxygen delivery. Preferably, this fitting is sufficiently snug so as to create an air-tight, near air-tight, or hermetic, seal. The other, or second ends, of the supply cannulae 42 are attached to an oxygen supply or source, such as a direct feed or an oxygen tank, which provides a supply of oxygen to the lumen of the supply cannulae and, ultimately, to the user of the apparatus 30. The length of the supply cannulae 42 may vary from very short to very long, as may be necessary to accommodate an individual user's oxygen supply and mobility needs.

The supply cannulae 38 may be of any type of standard, flexible medical tubing typically used to deliver oxygen from a source to a patient, such as tubing made of rubber, silicone, polyvinylchloride, polyurethane, plastic, Silastic®, nylon, fluoroplastic resin, polytetrafluoroethylene, fluorinated ethylene propylene, perfluoroalkoxy copolymer resin, ethylene tetrafluoroethylene, ethylene chlorotrifluoroethlyene, tetrafluoroethylene, hexafluoropropylene, vinylidene fluoride, or any similar form of polymer that may be formed into air-tight, flexible tubing, and the present invention is intended to encompass supply cannulae 38 made of all types of such tubing.

Preferably, the nasal cannulae 40 are connected to the proximal open ends 44 of the temple pieces 36 at locations along side a user's nose when the apparatus 30 is in use. The nasal cannulae 40 are connected to the proximal open ends 44 such that they may be at least partially inserted into the user's nostrils. Similar to the connection described above for the distal open ends 42, to facilitate the connection between the proximal open ends 44 and the nasal cannulae 40, the proximal open end portions 44 of the temple pieces 36 are configured to provide a point of attachment for the nasal cannulae 40, with the nasal cannulae 40 fitting snugly over points of connection at the proximal open end portions 44 such that a fluid connection is created between the nasal cannulae 40 and the proximal open end portions 44. In some embodiments, the points of connection at the proximal open end portions 44 are modified portions of the proximal ends of the temple pieces 36 having a decreased wall thickness. In other embodiments, the points of connection at the proximal open end portions 44 are short, tubular inserts that are of a diameter such that they may be partially inserted into the proximal open end portions 44, leaving a portion of them outside of the proximal open end portions 44. These short, tubular inserts may be permanently attached to the proximal open end portions 44 by any number of standard means, including without limitation, welding, soldering, adhesives, or other means described herein. Alternatively, the short, tubular inserts may be removable from the apparatus 30 such that they may be replaced when they become worn. In either embodiment, the modified portions of the proximal ends of the temple pieces 36 or the short, tubular inserts provide points of decreased diameter at the terminal points of the oxygen supply conduit of the temple pieces 36, over which first ends of the nasal cannulae 40 may be fitted to create a path of oxygen delivery. Preferably, this fitting is sufficiently snug so as to create an air-tight, or hermetic, seal. The other ends of the nasal cannulae 40 are preferably left open in order to provide an outlet from the apparatus 30 where oxygen may be delivered to a user.

In some embodiments, the nasal cannulae 40 are configured to have a 180-degree bend at the ends opposite the points of attachment to the proximal open ends 44 of the temple members 36, giving them a hook-like appearance. This feature serves to hook the nasal cannulae 40 under the lateral portion of the user's nostrils at the 180-degree bend such that at least a portion of the nasal cannulae 40 are located inside of the user's nostrils and are thus capable of delivering oxygen directly into the user's air passages. In that regard, it is preferable for the nasal cannulae 40 to be sufficiently rigid so as to be able to retain this hook-like shape and provide optimal oxygen delivery, while also being soft and pliable enough so as to provide comfort to the wearer of the apparatus 30 and may be made of any material suitable for this purpose, including any of the material the supply cannulae 38 may be made of. Preferably, the nasal cannulae 40 are made of a standard moderately rigid rubber, typically used for oxygen delivery and of sufficient density to permanently hold the hook shape but also of a sufficient pliability so as to provide comfort to the user of the device 30. The nasal cannulae 40 also serve to reduce the visual appearance of the oxygen tubing attached to the apparatus 30 of the present invention, which confers an aesthetic advantage over known oxygen delivery apparatuses that employ delivery tubes that curve under the entirety of the bottom of the user's nose, which are quite conspicuous on the user's face.

During operation, the oxygen supply or source is activated and configured to provide a desired amount of oxygen output. This oxygen output is transmitted from the supply or source directly to the supply cannulae 38, via the connection between the supply or source and the supply cannulae 38. The flow of oxygen proceeds through the supply cannulae 38 to the distal open end portions 42 of the temple members 36 where it passes into the lumen of the hollow tubular temple members 36 via the connection between the supply cannulae 38 and the distal open end portions 42. The oxygen then flows through the lumen of the hollow tubular temple members 36 to the proximal open end portions 44 of the temple members 36. At this point, the oxygen passes through the connection between the proximal open end portions 44 and the nasal cannulae 40 and into the nasal cannulae 40, where it is passed out of the apparatus 30 and is made available for the user. In some embodiments, the nasal cannulae 40 terminate inside of the user's nostrils, thus allowing the flow of oxygen to be delivered directly to the user's air passages.

Preferably, both the base frame 32 and the temple pieces 36 are made of a material that is sufficiently bendable or formable so as to allow the user to customize the fit of the apparatus 30 to his or her face, but that is also sufficiently rigid so that the bending or forming does not compromise the structural integrity of the base frame 32, the fully assembled apparatus 30, or the path of oxygen delivery provided by the lumen of the temple pieces 36. By way of example, and not limitation, the base frame 32 and/or the temple pieces 36 may be made of plastic, wire, or a lightweight alloy of a highly tensile metal, such as aluminum or titanium. Preferably, the base frame 32 and/or the temple pieces 36 are made of a lightweight, slightly bendable stainless steel. In some embodiments, the base frame 32 and the temple pieces 36 are made of the same material.

In an alternate embodiment, the base frame 32 and the temple pieces 36 are cast together as a single, integrated unit that includes a continuous, seamless tubular member located on each side of an eyeglass-shaped oxygen delivery apparatus that is capable of delivering oxygen to a user and also positioning lenses in front of the user's eyes. In this embodiment, the temple pieces 36 and the base frame 32 form the top portion of the base frame 32 and also secure the lenses 48, forming a continuous hollow tubular member that is in direct, fluid communication with the supply cannulae 38 and the nasal cannulae 40. The portion of the base frame 32 housing the lenses and the nose pads 34 are thus cast into the temple members 36 so that the apparatus 30 becomes a single hingeless and seamless unit.

Embodiments of the present invention may comprise any one or more of the novel features described herein, including in the Detailed Description, and/or shown in the drawings. The claims may include one or more features of any one or more of the embodiments described herein. For example, one or more features of one embodiment may be claimed in combination with one or more features of another embodiment, and no portion of this specification limits such claims.

The present invention, in various embodiments, includes components, methods, processes, systems and/or apparatuses substantially as depicted and described herein, including various embodiments, subcombinations, and subsets thereof. Those of skill in the art will understand how to make and use the present invention after understanding the present disclosure. The present invention, in various embodiments, includes providing devices and processes in the absence of items not depicted and/or described herein or in various embodiments hereof, including in the absence of such items as may have been used in previous devices or processes, e.g., for improving performance, achieving ease and\or reducing cost of implementation.

The foregoing discussion of the invention has been presented for purposes of illustration and description. The foregoing is not intended to limit the invention to the form or forms disclosed herein. In the foregoing Detailed Description for example, various features of the inventions are grouped together in one or more embodiments for the purpose of streamlining the disclosure. This method of disclosure is not to be interpreted as reflecting an intention that the claimed inventions require more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

Moreover, though the description of the invention has included descriptions of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An oxygen delivery apparatus, comprising:
a base frame configured to house a pair of lenses;
a first hollow tubular temple member having a distal open end and a proximal open end, and which is configured to be attached to a first portion of the base frame; and
a second hollow tubular temple member having a distal open end and a proximal open end, and which is configured to be attached to a second portion of the base frame;
wherein the first and second hollow tubular temple members and base frame do not include a hinge.

2. The apparatus of claim 1, further comprising supply cannulae connected to the distal open ends of the first and second hollow tubular temple members to form at least a substantially air tight seal, said cannulae being operable to deliver oxygen into the first and second hollow tubular temple members.

3. The apparatus of claim 1, further comprising nasal cannulae connected to the proximal open ends of the first and second hollow tubular temple members to form at least a substantially air tight seal, said cannulae being operable to deliver oxygen from the first and second hollow tubular temple members to a wearer of the apparatus.

4. The apparatus of claim 1, further comprising means for attaching the first hollow tubular temple member to the first portion of the base frame and means for attaching the second hollow tubular temple member to the second portion of the base frame.

5. The apparatus of claim 1, wherein the first hollow tubular temple member and the second hollow tubular temple member are seamless.

6. The apparatus of claim 1, wherein the base frame contacts only a portion of the lenses.

7. The apparatus of claim 6, wherein the portion of the lenses is the top portion.

8. The apparatus of claim 1, wherein the base frame contacts the entire perimeter of the lenses.

9. The apparatus of claim 4, wherein the means for attaching is selected from the group consisting of welding, soldering and adhesives.

10. The apparatus of claim 1, wherein the first hollow tubular temple member is attached to the first portion of the base frame at a single location and wherein the second hollow tubular temple member is attached to the second portion of the base frame at a single location.

11. The apparatus of claim 1, wherein the first hollow tubular temple member is attached to the first portion of the base frame at two locations and wherein the second hollow tubular temple member is attached to the second portion of the base frame at two locations.

12. The apparatus of claim 1, wherein the apparatus resembles a pair of eyeglasses.

13. An oxygen delivery apparatus, comprising:
an eyeglass frame comprising a base frame with a pair of lenses, and first and second hingeless hollow tubular temple members each including a proximal open end and a distal open end, wherein the first hollow tubular temple member is attached to a first portion of said base frame and the second hollow tubular temple member is attached to a second portion of said base frame;
first connector portions disposed at the distal end of the first and second tubular temple members to receive a supply cannula; and
second connector portions disposed at the proximal end of the first and second tubular temple members to receive a nasal cannula.

14. The apparatus of claim 13, wherein the first hollow tubular temple member and the second hollow tubular temple member are fixedly attached to the base frame.

15. The apparatus of claim 13, wherein the first connector portions comprise a reduced diameter of the distal end of the first and second tubular members and the second connector portions comprise a reduced diameter of the proximal end of the first and second tubular members.

16. The apparatus of claim 13, wherein the first connector portions comprise first tubular inserts at least partially inserted into the distal end of the first and second tubular members and the second connector portions comprise second tubular inserts at least partially inserted into the proximal end of the first and second tubular members.

17. The apparatus of claim 16, wherein the first tubular inserts are fixedly attached to the distal end of the first and second tubular members and the second tubular inserts are fixedly attached to the proximal end of the first and second tubular members.

18. The apparatus of claim 16, wherein the first tubular inserts are removable from the distal end of the first and second tubular members and the second tubular inserts are fixedly attached to the proximal end of the first and second tubular members.

19. The apparatus of claim 13, wherein the first and second hingeless hollow tubular temple members are seamless.

20. The apparatus of claim 13, wherein the lenses are corrective.
